(19) United States
(12) Patent Application Publication
(10) Pub. No.: US 2009/0156886 A1
(43) Pub. Date: Jun. 18, 2009

(54) METHOD AND APPARATUS FOR PROVIDING AUTOMATIC EYE FOCUSED THERAPY

(75) Inventors: PAUL A. BURGIO, ST. PAUL, MN (US); ANTHONY T. SPURRILL, EAGAN, MN (US); DAVID J. BURGIO, ST. PAUL, MN (US)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(73) Assignee: SYNAPSE RESEARCH COMPANY, ST. PAUL, MN (US)

(21) Appl. No.: 11/954,864

(22) Filed: Dec. 12, 2007

(51) Int. Cl. A61M 21/00

(52) U.S. Cl. 600/27; 607/45

(57) ABSTRACT

A method and system for providing therapy to a patient. A headset can be used that automatically provides stimulation to the eyes. A stimulator device may be worn over the chest that provides feedback data to a controller. The controller can analyze the received feedback data in order to update the lights and can dynamically alter the therapeutic display provided through the headset.

### FIELD OF THE INVENTION

This invention relates to the field of therapy. In particular, the invention relates to a method and apparatus for providing improved eye movement desensitization and reprocessing (EMDR) treatment and/or analogous treatment.

### BACKGROUND OF THE INVENTION

Eye Movement Desensitization and Reprocessing (EMDR) is a treatment that involves having an EMDR therapist instruct a patient to recall a picture from a traumatic event, or some negatively associated image and to identify related negative thoughts/beliefs. The patient is also instructed to notice associated body sensations, and to move their eyes rapidly back and forth. Throughout this procedure the patient is asked to provide their subjective level of disturbance. It has been found that after EMDR treatment, some patients report significantly reduced levels of symptoms related to a variety of psychological disorders such as anxiety or post traumatic stress disorder (PTSD).

There are a number of ways in which the EMDR procedure can be applied. One method in which it can be applied is through the usage of rapid eye movements. Other methods are similar to the treatment process described above except that they use different sensory stimuli than that of the eyes. For example, alternating tones may be used. A tone may be provided in the right ear, then the left ear, then right ear, then left ear, etc. of the patient. Another method involves the use of alternating tactile stimulation of the patient's hands, knees, shoulders, feet, of other body parts. For example, the therapist can gently touch the patient's right hand, left hand, right hand, left hand, and so forth. All EMDR procedure variations appear to produce beneficial treatment effects with varying levels of benefit,

Patients tend to have a preference for one mode of treatment versus another. For example, some patients prefer to process their traumatic memories with eyes closed, and some prefer processing trauma in a quiet environment. Some patients prefer the tactile stimulation because it helps them feel more grounded. Sometimes it is necessary to switch to tones or tactile stimulations in mid-session. It is recommended that EMDR therapists are flexible and open to the varying needs and preferences of patients when deciding which variation to use at a given moment. All variations may be useful and have a necessary place in EMDR therapy. EMDR therapy will now be discussed in more detail below.

As discussed above, EMDR integrates elements of different therapeutic methods in structured protocols that are designed to maximize the effects of treatment. These various methods include psychodynamic, cognitive behavioral, interpersonal, experiential, and body-centered therapies. EMDR is an information processing therapy and can use a multi-phase approach.

During EMDR, the patient attends to past and present experiences in brief sequential doses while simultaneously focusing on an external stimulus. Then the patient is instructed to let new material become the focus of the next set of dual attention. This sequence of dual attention and personal association is repeated many times in the session.

A first phase may be a history taking session during which the therapist assesses the client's readiness for EMDR and develops a treatment plan. The patient and therapist identify possible targets for EMDR processing. These include recent distressing events, current situations that elicit emotional disturbance, related historical incidents, and the development of specific skills and behaviors that will be needed by the patient in future situations.

During the second phase of treatment, the therapist ensures that the patient has adequate methods of handling emotional distress, good coping skills, and that the patient is in a relatively stable state. If further stabilization is required, or if additional skills are needed, the therapy may focus on providing these. The patient is then able to use stress reducing techniques whenever necessary, during or between sessions. However, one goal is not to need these techniques once therapy is complete.

In phases three through six, a target is identified and processed using EMDR procedures. These involve the patient identifying the most vivid visual image related to the memory, a negative belief about self, related emotions and body sensations. The patient also identifies a preferred positive belief. The validity of the positive belief is rated, as is the intensity of the negative emotions.

After these phases, the patient is instructed to focus on the image, negative thought, and body sensations while simultaneously moving his or her eyes back and forth following the therapist's fingers as they move across his or her field of vision for 20-30 seconds or more, depending upon the needs of the patient. Although eye movements are the most commonly used external stimulus, therapists often use auditory tones, tapping, or other types of tactile stimulation. The kind of dual attention and the length of each set are customized to the needs of the patient. The patient is instructed to just notice whatever happens. After this, the clinician instructs the patient to let his or her mind go blank and to notice whatever thought, feeling, image, memory, or sensation comes to mind. Depending upon the patient's report the clinician will facilitate the next focus of attention. In most cases a patient directed association process is encouraged. This is repeated numerous times throughout the session. If the patient becomes distressed or has difficulty with the process, the therapist follows established procedures to help the patient resume processing. When the patient reports no distress related to the targeted memory, the clinician asks him or her to think of the preferred positive belief that was identified at the beginning of the session, or a better one if it has emerged, and to focus on the incident, while simultaneously engaging in the eye movements. After several sets, patients generally report increased confidence in this positive belief. The therapist checks with the patient regarding body sensations. If there are negative sensations, these are processed as above. If there are positive sensations, they are further enhanced.

In phase seven, the therapist may ask the patient to keep a journal during the week to document any related material that may arise and reminds the patient of the self-calming activities that were mastered in phase two.

A follow up session can begin with an evaluation of the previous session, and of progress since the previous session. EMDR treatment ensures processing of all related historical events, current incidents that elicit distress and future scenarios that will require different responses. The overall goal is to produce the most comprehensive and profound treatment effects in the shortest period of time, while simultaneously maintaining a stable patient within a balanced system.

After EMDR processing, patients generally report that the emotional distress related to the memory has been eliminated, or greatly decreased, and that they have gained important cognitive insights. Importantly, these emotional and cognitive changes usually result in spontaneous behavioral and personal change, which are further enhanced with standard EMDR procedures.

Although EMDR has proven effective in the past there are still limiting factors related to those physiologic responses that are not under a patient's conscious control. Much of the protocol is determined by the patient's perception or on the patient's direct feedback. This can be variable and a self limiting factor for success. Therefore there is a need in the field to overcome these limiting factors by developing a system and method note that this is now that that can detect those measurable physiological features while providing improved therapeutic treatment.

### SUMMARY OF THE INVENTION

It is therefore an object of the invention to provide improved therapeutic means to a patient.

Another object of the invention to provide automatic means for providing therapy to a patient.

Yet another object of the invention can be a system for providing therapy to a patient that uses visual stimulation or other sensory modalities that are used individually or in combination.

Still yet another object of the invention can be a system for providing therapy to a patient that uses tactile stimulation.

In accordance with these and other objects of the invention, an aspect of invention can be a system for providing therapy to a patient comprising: a headset having visual stimulating means for providing visual stimulation to a patient; a stimulator having tactile stimulating means for providing tactile stimulation to a patient; and a programmable controller operably connected to the visual stimulating means in order to provide instructions for providing visual stimulation to the patient.

Another aspect of the invention can be a method for providing therapy to a patient comprising: performing a pre-treatment diagnosis of a patient; developing a treatment plan for the patient; programming a headset and a stimulator to function in accordance with results from the pre-treatment diagnosis; and using the headset and the stimulator with the patient in order to provide treatment.

These and various other advantages and features of novelty that characterize the invention are pointed out with particularity in the claims annexed hereto and forming a part hereof. However, for a better understanding of the invention, its advantages, and the objects obtained by its use, reference should be made to the drawings which form a further part hereof, and to the accompanying descriptive matter, in which there is illustrated and described a preferred embodiment of the invention.

### BRIEF DESCRIPTION OF THE DRAWINGS

FIG. 1 is a front view of a headset in accordance with an embodiment of the present invention.

FIG. 2 is a rear view of the headset shown in FIG. 1.

FIG. 3 is a front view of a stimulator in accordance with an embodiment of the present invention.

FIG. 4 is a schematic showing the headset and stimulator communicating with each other.

FIG. 5 is a schematic of a transducer assembly in accordance with an embodiment of the present invention.

FIG. 6 is a front view of a pen in accordance with an embodiment of the present invention.

FIG. 7 is a diagram of a system in accordance with an embodiment of the present invention.

FIG. 8 is a flow chart of a method for providing the therapeutic treatment, in accordance with an embodiment of the present invention.

FIG. 9 is a flow chart of the application of therapeutic treatment process to a patient using the therapeutic system, in accordance with an embodiment of the present invention.

### DETAILED DESCRIPTION OF THE INVENTION

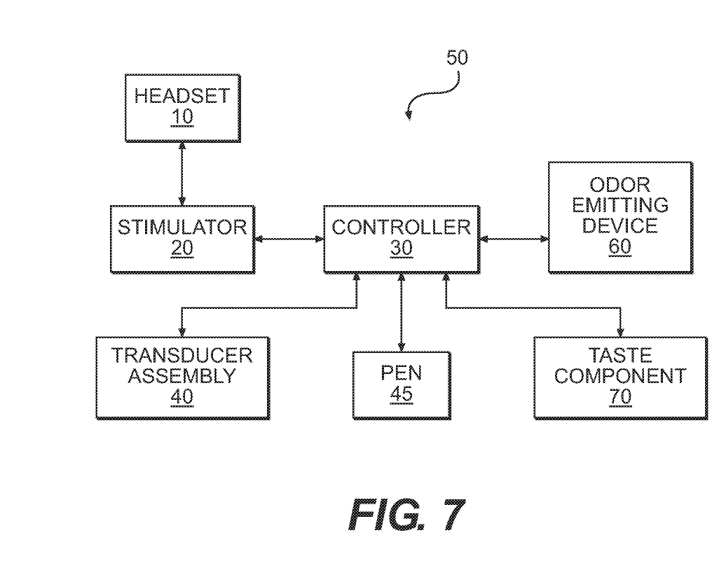

The present invention is a method and system for providing therapy to a patient, preferably EMDR therapy, or a therapy similar to EMDR. The method and system overcomes among other things the limitation of not being able to monitor and respond to physiological responses by utilizing devices that monitor and respond to these behaviors. The present method and system also engages multiple areas of a patient's brain while using multi-sensory feedback and feed forward mechanisms. This system permits usage for physiologic disorders and for lifestyle enhancement. This process can have an effect on the mental, physical, psychological and biochemical functions of a patient through the usage of uni-modal and/or multi-modal methods specific to the diagnostic findings. The therapeutic system 50, shown in FIG. 7 and discussed in more detail below, is capable of reproducing the clinical treatment that is provided by a therapist.

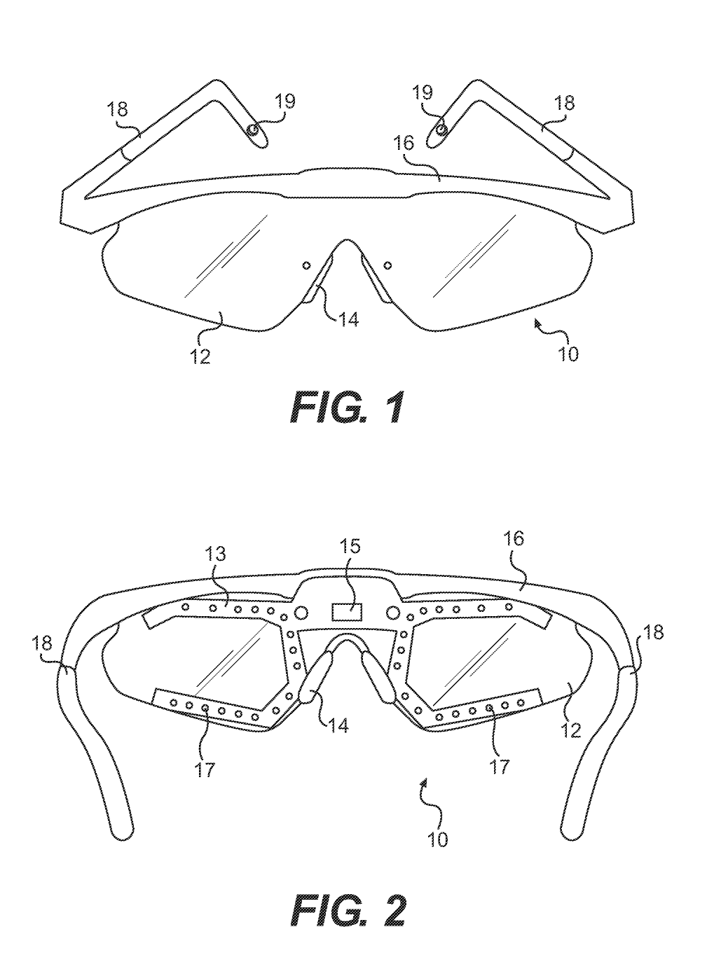

Now turning to those components that make up the therapeutic system 50 and specifically to FIG. 1, where a front view of an embodiment of a headset 10 that may be used in the performance of the present invention is shown. The headset 10 in the embodiment shown is a pair of glasses; however it should be understood that the headset 10 may be constructed out of other devices that are able to be attached to the head, such as helmets, hats, goggles, etc. The headset 10 has a frame 16 having a pair of arms 18 that extend from the frame 16 and hold the headset 10 on the patient's ears. A nose rest 14 is further attached to the headset 10 and enables the headset 10 to be positioned on the patient's nose. A pair of lenses 12 is held by the frame 16. Preferably the lenses 12 are darkened or block out external light to some degree, if not totally, however the lenses 12 may be clear or absent in some embodiments.

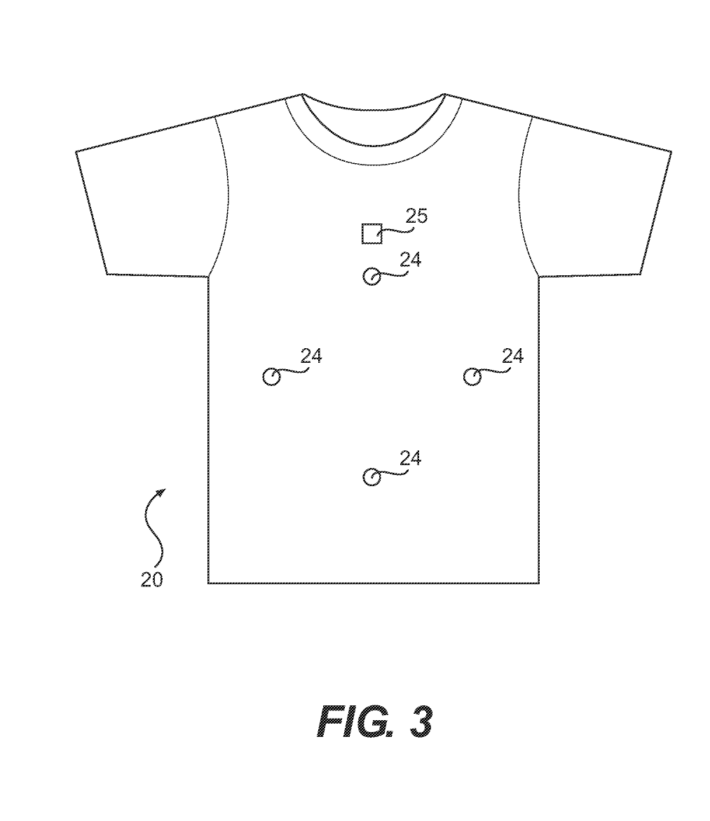

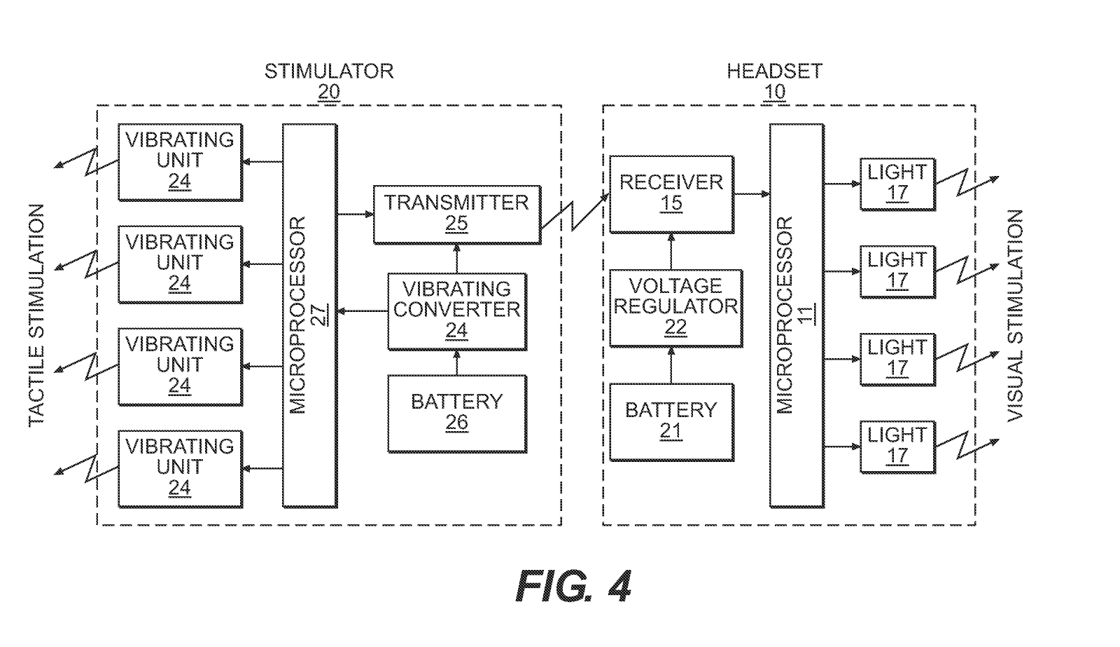

Referring now to FIG. 2, wherein a rear view of the headset 10 is shown, attached to the frame 16 is a receiver 15, in some embodiments the receiver 15 may instead be replaced with a transceiver. The receiver 15 is able to receive signals from an external device. In the embodiment shown, the receiver 15 is wireless and receives the signals via infrared; however the headset 10 may be directly wired to an external device, such as a central controller 30 shown in FIG. 7, or to the stimulator 20, shown in FIGS. 3 and 4, in order to receive commands for controlling the stimulation that is provided to the patient. A microprocessor 11 (shown in FIG. 4) and/or data storage device may be embedded and/or attached to the frame 16 or some other part of the headset 10. The microprocessor 11 and data storage device may be a microchip, or circuit board, or some other device that may have preprogrammed and/or programmable features.

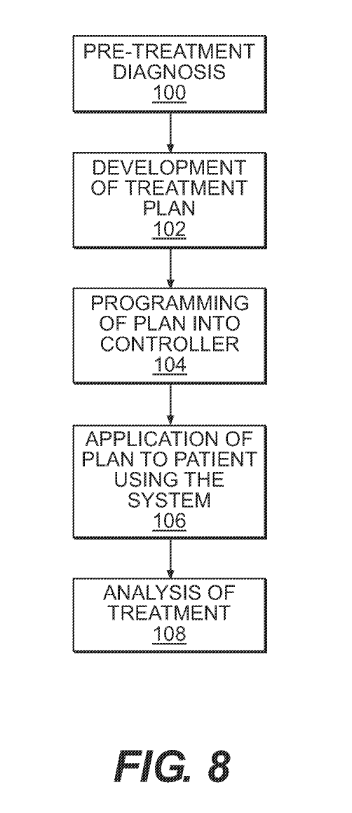

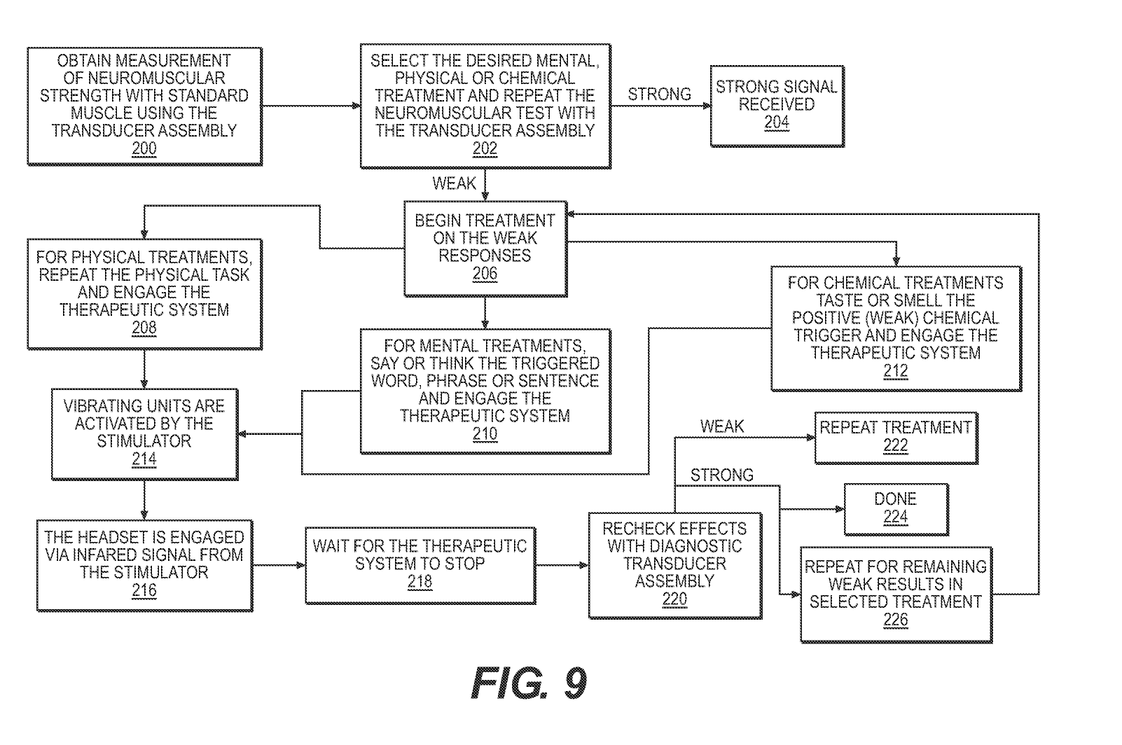

Still referring to FIG. 2, additionally attached to the frame 16 is a light frame 13 that supports a number of lights 17. The lights 17 may be LEDs or some other miniature lighting device. The lights 17 are shown arranged in a manner that places them along the periphery of the lenses 12. In alternative embodiments the lights 17 may fill the entire field of view. In yet other embodiments, the lenses 12 may further have LCD screens or fill emersion goggles that can provide pictures, videos and/or various shapes and colors. The lights 17 may be activated in a preprogrammed sequence and/or controlled remotely. The control signals may be sent through the receiver 15 and/or the headset 10 may use the internal memory and microprocessor 11 that has pre-programmed sequences stored. The lights 17 provide visual stimulation to a patient and are used as part of the treatment process, which is discussed in more detail below with reference to FIGS. 8 and 9.

It is also possible to have in some embodiments, audio devices 19 that may be embedded in the aims 18 and be able to provide auditory stimulation to a patient in addition to visual stimulation. The audio devices 19 may be better incorporated in a larger headset, such as a helmet or goggles.

Now turning to FIG. 3, a front view of a stimulator 20 in accordance with an embodiment of the present invention is shown. The stimulator 20 is designed to provide tactile stimulating means. The stimulator 20 is composed of one of more types of vibrating units 24 embedded and/or attached to a shirt, vest, jacket of similar article of clothing which is worn on the torso. The shirt (e.g. body armor) can be stretchable so as to hold the vibrating units 24 against the chest. The vibrating units 24 may be located near specific points on a patient's chest when the stimulator 20 is worn. In the embodiment shown in FIG. 3 the vibrating units 24 are located along the sternum, about an inch above the umbilicus and midway between the second third ribs bilaterally. The vibrating units 24 may consist of vibrating motors, piezoelectric vibrators, electric solenoids, etc.

Still referring to FIG. 3, an infrared transmitter 25 is mounted on stimulator 20 over the sternum and can communicate with the receiver 15 that is part of the headset 10. It is also possible that in some embodiments, transmitter 25 is replaced with a transceiver that is also able to receive signals. When employing a transceiver, the stimulator may also communicate with a central controller 30, shown in FIG. 7, which can transmit control signals. The stimulator 20 may provide selective patterns of mechanical stimulation to specific points on the body. In the embodiment shown in FIGS. 1-4, stimulation may be coordinated with eye movements coordinated with the headset 10. The stimulator 20 also has a microprocessor 27 and storage device that can have pre-programmed control signals stored therein or be programmable.

FIG. 4 is a schematic showing the headset 10 and stimulator 20 communicating with each other, in accordance with an embodiment of the invention. Stimulator 20 is shown providing tactile stimulation through the vibrating units 24. Also shown is the microprocessor 27, which is acting as a controller for the stimulator 20 and providing instructions to the vibrating units 27 and instructions to the transmitter 25. A battery 26 and voltage converter 23 are used to provide energy to the transmitter 25, the microprocessor 27 and the vibrating units 24. The transmitter 25 transmits signals which are received at the headset 10 via the receiver 15. The signals carry instructions which are provided to the microprocessor 11, which acts as a controller for the headset 10. The instructions provide the sequences in which the lights 17 should be activated in order to provide visual stimulation to a patient. The battery 21 and voltage regulator 22, provide energy to the receiver 15, the microprocessor 11 and lights 17.

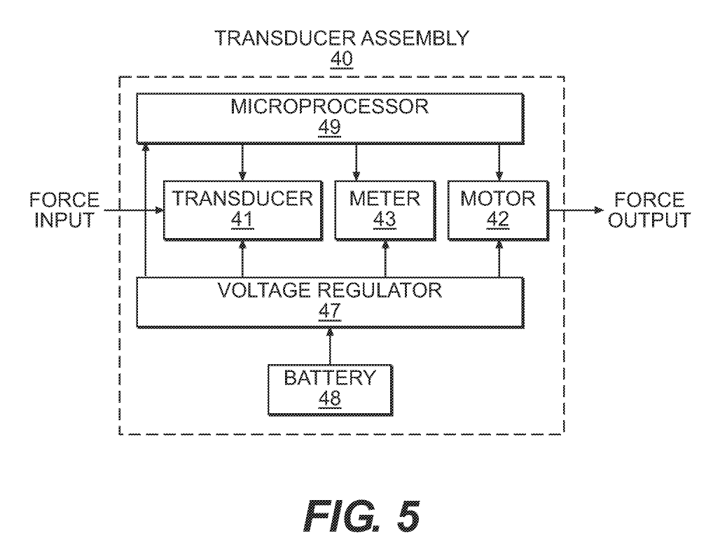

Now turning to FIG. 5, a schematic of a transducer assembly 40 in accordance with an embodiment of the present invention is shown. The transducer assembly 40 is a diagnostic device that may have a transducer 41, a motor 42 and a meter 43. The force transducer 41 measures the force produced by a patient's muscle being tested. The electric motor 42 produces a specific amount of force in order to test the function of the patient's muscle. The transducer 41 and the motor 42 may be mounted in parallel with two anchor points. The diagnostic function of the transducer assembly 40 is achieved by attaching one end of the force transducer 41 and the electric motor 42 to the patient's arm or leg that is being tested. The other end of the transducer assembly may be attached to a stationary object, such as the opposite leg of the patient. The battery 48 and the voltage regulator 47 provide energy to the transducer 41, the meter 43 and the motor 42, as well as the microprocessor 49. The microprocessor 49 operates as a controller for the transducer assembly 40 and provides instructions to the device.

The transducer assembly 40 may optionally have a meter 43 that is used to provide visual indication as to the total force generated and whether or not the muscle is strong or weak. Alternatively, signals may be transmitted to a central controller 30, which may then in turn display the received readings. When using a meter 43 the total force received in pounds may be displayed and a message indicating the condition of the muscle such as “strong” or “weak” may be displayed. This information can help to determine the treatment protocols for optimal clinical benefit.

When used the transducer assembly 40 may measure the maximum force of a patient's muscle after the start of a sequence and monitor force throughout the sequence. The transducer assembly 40 may further calculate the values for 50%, 75% and 115% of the maximum force. The transducer assembly 40 may then apply the necessary power to the motor 42 in order to generate these forces for 0.5 sec at each force. During the application of the method, 2 repetitions of applying the three different forces discussed above may be performed. The maximum required force of the motor 42 is 25-35 lbs for most patients.

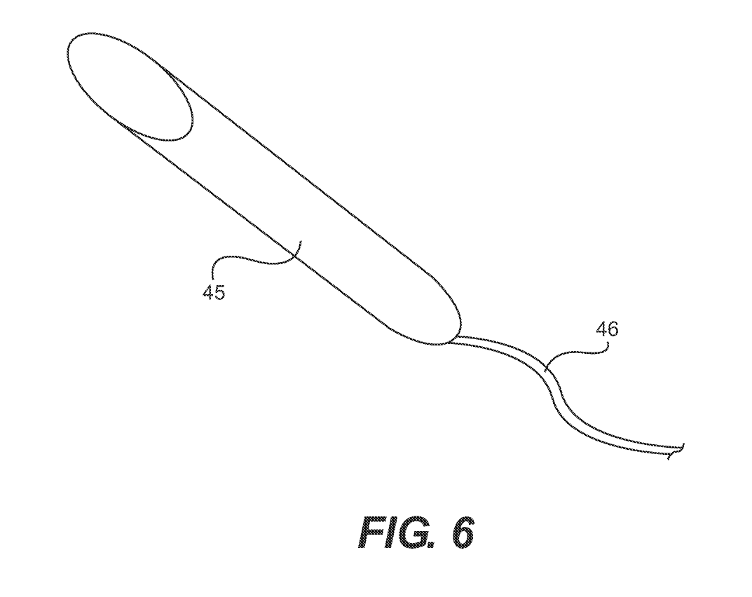

The transducer assembly 40 may use the microprocessor 49 to perform all necessary measurements, calculations and controls. Alternatively, or in addition to microprocessor 49, the controller 30, shown in FIG. 6, may perform the necessary calculations and controls of the transducer assembly 40 as well as the other components. When using the controller 30, communication between the controller 30 and the transducer assembly 40 may occur wirelessly and/or through wires.

Now turning to FIG. 6, a pen 45 is shown that may be used in an embodiment of the present invention. The pen 45 may be adapted to provide various different types of stimulation, simultaneously and/or independently with other components if desired. The pen 45 may emit audio information, light stimulation or different colors, and/or vibrate to provide tactile stimulation. Specifically the pen 45 can stimulate individual muscle groups and mechanoreceptors over the body through a series of motors that can be embedded in the pen 45. The muscles stimulated depend upon the source of the identified clinical weakness. These various forms of stimulation can improve muscle strength and influence central cognition or improve central logic communication. A wire 46 extends from the pen 45 and may be connected to the controller 30. Alternatively, the pen 45 may have microprocessor and storage device embedded within it that is preprogrammed to perform a number of operations.

In FIG. 7 a diagram of an embodiment of a therapeutic system 50 used to perform the present invention. The therapeutic system 50 is designed to be an automated system in order to provide improved results in therapy. The therapeutic system 50 is shown with the headset 10, the stimulator 20, the controller 30, the transducer assembly 40, the pen 45, an odor emitting device 60 and a taste component 70. Although shown with these components, the method of the present invention is typically practiced with fewer than the shown components. It should further be known that the components may be employed at different times and places. Typically, only the headset 10, the stimulator 20, the controller 30 and the transducer assembly 40 are used. Although in some embodiments only the transducer assembly 40, the headset 10 and the stimulator 20 may be used. The headset 10 and the stimulator 20 may directly communicate with each other, while the transducer assembly 40 is used for diagnostic and post-therapeutic processes.

In the shown therapeutic system 50 a central controller 30 is used to communicate with the various components of the system. This may be accomplished either wirelessly or through wired connections with each of the components. The controller 30 may be a processor or array of processors and data storage devices, such as personal computers and other computing devices both portable and non-portable. In some embodiments, the controller 30 may be located at a non-local location and be accessed via the Internet and/or wirelessly. The controller 30 is capable of being programmed in order to provide commands to the various components of the therapeutic system 50. The controller 30 is also able to receive data from the various components and analyze the received data. It should be understood that while the therapeutic system 50 shown in FIG. 6 uses a central controller 30, the functions and capabilities of the controller 30 may be distributed to each of the components individually. In embodiments not using a central controller 30, the various components of the system may communicate with each other. When distributed, the various components may also perform the analytic processes.

Additionally shown in FIG. 7 is the taste component 70 and the odor emitting device 60. These devices may optionally be part of the therapeutic system 50 and are shown here in order to illustrate that devices dedicated to each of the senses may be used when performing the method of the present invention. The odor emitting device 60 may release certain odors depending upon what is needed in order to perform the therapy. The taste component 70 may release certain tastes based upon what is needed in order to perform the therapy. These components may be automated and in communication with the controller 30 much in the way the other components of the therapeutic system 50 are and/or alternatively have internal microprocessors that are capable of controlling the components.

Now turning to the method performed using the therapeutic system 50, reference is made to FIG. 8 where a flow chart illustrating the steps performed in a therapeutic session is shown.

In step 100 a pre-treatment diagnosis is performed. The pre-treatment diagnosis is to measure neural reactivity of a patient by using the transducer assembly 40 to measure the force generated by the muscle and then to apply a counterforce to measure the patient's ability for neuromuscular recruitment. Other devices that can measure muscle reactivity besides the transducer assembly 40 may be used as well. Measuring the neuromuscular recruitment identifies subtle neurological changes that indicate the specific treatment plan that should be applied to the patient. Reactivity can be measured using other diagnostic means than the transducer assembly 40, such as temperature, heart rate, and other autonomic functions and/or other physical task oriented behavior. The diagnostic means, in particular the transducer assembly 40, can also be used in a diagnostic procedure to correct mental and physical stress. The stimulator 20 can then be used therapeutically to correct poor results discovered in the pre-treatment diagnosis.

The pre-treatment diagnostic procedure is also used to determine the treatment schedule. Part of the pre-treatment diagnostic can be a web-based or office-based questionnaire in order to determine the treatment protocol to prescribe. The diagnostic may also include a determination of the physical, chemical and mental stimuli that produce a muscle weakness. These stimuli can be presented by any component of the device or by the practioner.

In general, when performing the diagnostic using the transducer assembly 40 the more muscle groups used in the test the less sensitive the test results are. Results are more sensitive when a single muscle group is selected for testing. Generally testing a single muscle group is preferred to increase sensitivity. Preferred muscles are the rectus femora's or iliacus muscles.

In step 102 a treatment plan is developed based upon the results of the pre-treatment diagnosis. The treatment plan is directed towards providing instructions to the usage of various components of the therapeutic system 50. The treatment plan can be developed based upon automatic analysis of the pre-treatment diagnosis results.

In step 104 the plan can be programmed into a controller, such as controller 30 discussed above. The controller 30 may be operably connected to the components of the therapeutic system 50 in order to provide instructions. The instructions are then provided to the components, such as the headset 10 and the stimulator 20. Alternatively, the components of the therapeutic system 50 may be programmed directly and the controller functions distributed to the various components.

The plan may additionally be downloaded to a patient's home computer that can be linked with a website of the therapist in order to accurately update, assess, and treat the patient. This Internet based treatment provides an option for home based mental, chemical, and physical stress. The availability of home based treatment for patients assists in reducing the workload for healthcare professionals.

In step 106 the plan is applied to the patient via the components of the therapeutic system 50. For example, the treatment plan may involve the stimulation of between 4 to 12 points on the chest using the stimulator 20 while simultaneously shifting the gaze of patient in specific patterns. The pattern may be moving the eyes in an upper right direction, a lower left direction, repeating and then to upper left direction and lower right direction, then repeating. It is to be understood that various other eye patterns may be used and the patterns chosen may be based upon the therapist's experience with EMDR.

In step 108 the results of the application of the plan to the patient are then analyzed. The results are used in identifying physiological weakness in conjunction with statements, thoughts or physical, chemical or energetic stimuli. A patient's muscle weakness is used as an indicator to provide immediate identification of a weakness and also for an immediate indicator of the effectiveness of the treatment. This can be accomplished by using the transducer assembly 40 in conjunction with the other components of the therapeutic system 50.

The treatment plan may involve various components of the system and may use various methodologies and types of stimulation. An example of a treatment plan may involve using efferent copy (sending a copy to other parts of the nervous system) feedback and feed forward mechanisms in order to accomplish results. For example a wobble board may be used by the patient while using the therapeutic system 50. The sensory stimulation and/or exercise provide additional stimulation to the patient. Using mental and physical stimulations and/or exercises can increase feedback to the previously measured areas of the patient's brain that were determined to be deficient. The therapeutic system 50 and method develops and strengthens the association and communication of the body parts of the patient.

Auditory stimulation may also be part of the treatment plan. Auditory directed programs can be used to guide the conscious thought patterns so that they are in alignment with the treatment program. Additionally, subliminal positive affirmations can be used. These subliminal affirmations may be auditory affirmations that are provided during chest and head stimulation. Directing a patient's thoughts can enhance the treatment program. Prior to treatment the patient can complete a questionnaire that profiles their communication style, emotional status as well as perception of self and world. Also, the results will help identify the emotional and physical tone of the person. Through the questionnaire other needs of the patient and other potential detractors of clinical success can be identified, e.g. allergies, chemical toxicity, hormone imbalance, detrimental life style habits, past physical and emotional traumas. An auditory directed program can be presented via, MP3, downloaded from the internet, or on CD/DVD. It may be provided through the headset 10 or alternatively listened to through some other audio device, such as a CD player, or MP3 player.

Visual stimulation may also be provided as part of the treatment plan. The visual stimulation is provided in order to focus the direction of the eyes and also to activate specific areas of the brain. To focus the direction of the eyes a variety of light sources can be used but the preferred form is a point light source, such as that provided by the headset 10. To activate specific areas of the brain the light source can be of various colors, intensity, frequency of stimulation and shapes including pictures and images to invoke different physiological reaction. Movies of known and unknown content can be used. Visual stimulation can be provided using a variety of devices, such as the headset 10, i.e. glasses fitted with LED light of selected color and arranged in different patterns.

Also part of the treatment plan can be tactile stimulation that can be provided through usage of the stimulator 20. Stimulation can be provided to areas such as neural lymphatic points, which are intercostals and circumscribe the umbilicus. These points can be stimulated through gentle massage, vibration, thermal, laser, acupuncture needles or electrical stimulation (TENS). Additional points of stimulation include but are not limited to the following: Points on the back, including intercostals and Para spinal points; points on the head, such as the forehead, temporal, parietal, occipital and mastoid areas and the ears; points on the feet; points on the pubic bone and acupuncture meridian points on the arms and legs.

It is also possible to use olfactory stimulation and taste stimulation. This would involve providing specific odors and tastes in conjunction with memories and feelings.

Now turning to FIG. 9, where a flow chart illustrating an exemplary application of the therapeutic method to a patient using the therapeutic system is described.

In step 200, measurements of neuromuscular strength with standard muscle are obtained by using the transducer assembly 40 discussed above. This is done in order to procure a control value for the patient's muscular strength.

In step 202, the desired mental, physical or chemical treatment is selected by the therapist. The neuromuscular test with the transducer assembly 40 is repeated while using the selected treatment. Those signals that are strong are noted and those signals that are weak are noted.

In step 204, if a strong signal is received it is noted and indicated as an area that does not need treatment. In step 206, those weak responses that were noted are prepared for treatment.

In step 208, for physical treatments a patient repeats a physical task and the therapeutic system is engaged. In step 210, for mental treatments, the patient says or thinks the triggered word, phrase or sentence and the therapeutic system is engaged. In step 212, for chemical treatments, the patient tastes or smells the positive (weak) chemical trigger and engages the therapeutic system. The appropriate treatment is employed and the method moves onto to step 214.

In step 214, the vibrating units 24 are activated by the stimulator 20. In step 216, the headset 10 is engaged via infrared signal from the stimulator 20. The stimulator 20 and headset 10 perform a given pattern or sequence based upon the predetermined treatment plan.

In step 218, the therapist waits for the therapeutic system to stop. In step 220, the patient is checked again with the transducer assembly 40 in order to determine if any progress was made. For those muscles that provide weak signals, in step 222 the treatment is repeated. In step 224, the treatment is finished for those that provide strong signals. In step 226, the treatment is provided again to those muscles that provided weak signals.

The therapeutic system 50 is different from the standard EMDR due to the automation of the process. EMDR is a hands-on treatment program that is time sensitive and based on subjective markers whereas the therapeutic system 50 is objective and enables therapists to work with hundreds of patients at once. This is more cost effective and enables people to take control of their own health, as well as offering more specific measuring tools for progress of treatment. Unlike EMDR the method and system of the present invention can also be used for business related stressors that minimize job performance without losing time to travel to a doctor or take time off from work. Also unlike EMDR this can be used for treatment of physical injuries in the privacy of their own home, sporting facility, or any treatment facility.

Additional uses of the therapeutic system 50 may be for the treatment of Life Accumulation Stress Disorder (LASD). It has been observed that as people age they carry memories of life stressor's. It's these negative memories that “slow” people down in life, and is a reason why our performance may decline due to aging. Usage of this therapeutic system 50 may ameliorate this disorder.

The system and method can also be used to bring balance to the overall physical and mental well-being of the patient, which in turn enables the patient to make better choices, communicate more effectively in relationships, and increase physiological health through reduced stress. For example, fibromyagia, chronic fatigue, depression, back and neck pain, headaches, recovery time from sports injury, and anxiety can be treated. The method and system of the present invention reprograms thoughts and movements of the body so that it is not perceived as negative. As a result the environment is not controlling the body's responses. Retraining of conscious, subconscious and physical memory can allow the brain to function at its highest potential. The overall method and system decreases stress load and allows for improved physiological and mental healing processes.

Further uses may include, but are not limited to: post traumatic stress treatment; performance enhancement in the work force/sales; treatment for depression/non pharmaceutical; treatment for anxiety/non pharmaceutical; analysis of muscle weakness; analysis of neuromuscular weakness; identification of subconscious belief systems causing a physical consequence; identification of subconscious memories causing a physical consequence; physical exam of muscle and nerve systems done remotely; mood enhancement; performance enhancement/athletic/non pharmaceutical; treatment for other brain disorders like OCD, ADD, Bipolar; strength training; treatment for disorders like asthma, deconditioned syndrome; treatment for food, alcohol, gambling, sex, or any other addiction; identification of food sensitivities that may lead to a functional decline in overall health; and identification of automatic negative thoughts or stressful words unique to the individual and for mental program to build self worth.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. A method for providing therapy to a patient comprising:
performing a pre-treatment diagnosis of a patient;
developing a treatment plan for the patient;
programming a headset and a stimulator to function in accordance with results from the pre-treatment diagnosis; and
using the headset and the stimulator with the patient in order to provide treatment.

2. The method of claim 1, wherein the pre-treatment diagnosis is performed using a transducer assembly.

3. The method of claim 2, wherein the transducer assembly comprises a meter.

4. The method of claim 1, wherein the headset comprises a plurality of lights.

5. The method of claim 3, wherein programming the headset comprises determining a pattern of turning the plurality of lights on and off.

6. The method of claim 3, wherein the headset further comprises a transceiver.

7. The method of claim 1, wherein the headset further comprises an audio playing device.

8. The method of claim 1, wherein the treatment plan is based on EMDR.

9. The method of claim 1, wherein the treatment comprises providing visual stimulation and tactile stimulation.

10. The method of claim 1, wherein performing a pre-treatment diagnosis comprises detecting the neuromuscular recruitment.

11. A system for providing therapy to a patient comprising:
a headset having visual stimulating means for providing visual stimulation to a patient;
a stimulator having tactile stimulating means for providing tactile stimulation to a patient; and
a programmable controller operably connected to the visual stimulating means in order to provide instructions for providing visual stimulation to the patient.

12. The system of claim 11, wherein the system further comprises a transducer assembly.

13. The system of claim 12, wherein the transducer assembly comprises a meter.

14. The system of claim 11, wherein the headset comprises a plurality of lights.

15. The system of claim 11, wherein the headset comprises one or more LCDs.

16. The system of claim 11, wherein the headset further comprises a transceiver.

17. The system of claim 11, wherein the headset further comprises an audio playing device.

18. The system of claim 11, wherein the stimulator comprises a transceiver.

19. The system of claim 11, wherein the stimulator comprises one or more motors.

20. The system of claim 11, wherein the stimulator and the headset are operably connected.

21. The system of claim 11, wherein the programmable controller is operably connected to the tactile stimulating means.

22. The system of claim 1, wherein the controller is programmed with a treatment plan based on EMDR.
